(19) United States
(12) Patent Application Publication
(10) Pub. No.: US 2025/0009522 A1
(43) Pub. Date: Jan. 9, 2025

(54) ELBOW JOINT PROSTHESES

(71) Applicant: HOWMEDICA OSTEONICS CORP., Mahwah, NJ (US)

(72) Inventors: Matthew Victor KARTHOLL, Fort Wayne, IN (US); Michel HASSLER, Saint Ismier (FR)

(73) Assignee: HOWMEDICA OSTEONICS CORP., Mahwah, NJ (US)

(21) Appl. No.: 18/261,318

(22) PCT Filed: Jan. 20, 2022
(86) PCT No.: PCT/US2022/070268

§ 371 Date: Jul. 13, 2023

Related U.S. Application Data
(60) Provisional application No. 63/139,911, filed on Jan. 21, 2021.

(51) Int. Cl. A61F 2/40; A61F 2/30; A61F 2/38

(52) U.S. Cl. CPC A61F 2/4014; A61F 2002/30301; A61F 2002/30616; A61F 2002/3831

(57) ABSTRACT

An ulnar implant configured for implantation at an end portion of an ulna. The ulnar implant may be configured to articulate with a portion of a humerus, which may be a native end surface of the humerus bone or a humeral implant. The ulnar implant may have a curved or saddle shaped member. The curved or saddle shaped member may include a first concave surface and a second convex surface.

### FIELD OF DISCLOSURE

This application relates to an elbow joint prostheses and methods for assembling, fitting and implanting the same.

### BACKGROUND

Elbow prostheses are sometimes implanted in patients with deteriorating elbow joint function. The elbow joint function can deteriorate for a number of reasons, including osteoporosis, cartilage wear, trauma, and other reasons. The elbow is a complex joint involving three bones, with the joint being formed where the distal end of the humerus and the proximal ends of the radius and ulna meet. These bones are smaller than bones found at other more commonly replaced joints. As a result, the individual components are also smaller. Because the lower arm is highly mobile, artificial elbow joint components must be highly mobile and able to sustain a wide variety of loads without failing.

### SUMMARY

Some elbow prostheses can replace all three bone surfaces in the joint in a total elbow arthroplasty. Total elbow arthroplasty components can suffer from problems including loosening, disengagement, wear, and fracture of the components. It can therefore be advantageous to only resurface an articular surface or replace a portion of the elbow joint. This approach may be called elbow hemiarthroplasty. In an elbow hemiarthroplasty, the native articular surface of one or more of the distal humerus, proximal ulna, and proximal radius is or are retained while at least one native articular surface is treated, such as by placing an articular surface component over or in place of the native surface. As a result, the individual artificial components in an elbow hemiarthroplasty may articulate with the native bone. With fewer components implanted, the risk of loosening, disengagement, wear and fracture is lessened.

For the foregoing and other reasons, improved elbow joint prostheses and components therefore are desired. Some aspects of the present disclosure are directed to an ulnar implant.

According to some embodiments, provided is an ulnar implant comprising:
a curved body having a first end and a second end, the curved body comprises: a first surface; and
   a second surface, wherein the first surface and the second surface are on opposing sides of the curved body;
wherein the first surface has a concave curvature in a cross-sectional view when a section is taken along a first plane that intersects both S-I axis and A-P axis, wherein the S-I axis is an axis that extends in superior-inferior direction with respect to the curved body of the ulnar implant and the A-P axis is an axis that extends in antero-posterior direction with respect to the curved body of the ulnar implant;
wherein the second surface has a convex curvature in a cross-sectional view when a section is taken along the first plane, and
wherein when the ulnar implant is implanted at an end portion of an ulna, the first surface of the ulnar implant articulates with a portion of a humerus.

Also provided is a method for treating an elbow joint, comprising:
accessing an end portion of an ulna;
positioning an ulnar implant comprising a curved body at the end portion of the ulna, wherein the curved body having a first end and a second end, the curved body comprising:
   a first surface; and
   a second surface, wherein the first surface and the second surface are on opposing sides of the curved body;
   wherein the first surface has a concave curvature in a cross-sectional view when a section is taken along a first plane that intersects both S-I axis and A-P axis, wherein the S-I axis is an axis that extends in superior-inferior direction with respect to the curved body of the ulnar implant and the A-P axis is an axis that extends in antero-posterior direction with respect to the curved body of the ulnar implant;
   wherein the second surface has a convex curvature in a cross-sectional view when a section is taken along the first plane,
   wherein when the ulnar implant is implanted at an end portion of an ulna, the first surface of the ulnar implant articulates with a portion of a humerus; and
positioning the first surface of the ulnar implant at the end portion of the ulna to articulate with a portion of a humerus.

Some aspects of the present disclosure are directed toward a method for treating an elbow joint. The method can include accessing an end portion of an ulna, which can include forming a recess shaped to receive the ulnar implant in the end portion of the ulna. The method can include positioning an ulnar implant including a curved member at the end portion of the ulna. When the ulnar implant is positioned at the end portion of the ulna, a first concave surface of the ulnar implant can be configured to articulate with a portion of a humerus, such as a native humerus or a humeral implant. In some configurations, the articular surface can be configured to rotate relative to the portion of the humerus. When the ulnar implant is positioned at the proximal end portion of the ulna, a trochlea of the humerus and/or soft tissues of the elbow joint can at least partially constrain the ulnar implant in place. In some configurations, the ends of the curved member can be held in place by edges of a trochlear notch of the ulna, for example, the ends of the curved member can be held in place by a coronoid process and an olecranon process of the ulna. In some configurations, the ulnar implant can include pyrocarbon.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

These and other features, aspects and advantages are described below with reference to the drawings, which are intended for illustrative purposes and should in no way be interpreted as limiting the scope of the embodiments. Furthermore, various features of different disclosed embodiments can be combined to form additional embodiments, which are part of this disclosure. In the drawings, like reference characters denote corresponding features consistently throughout similar embodiments. The following is a brief description of each of the drawings.

FIG. 1 is an ulnar implant implanted in an elbow joint.

FIG. 2A is a perspective view of the ulnar implant shown in FIG. 1.

FIG. 2B is a side view of the ulnar implant of FIG. 2A.

FIG. 2C is another perspective view of the ulnar implant of FIGS. 2A and 2B.

FIG. 2D is a front view of the ulnar implant of FIGS. 2A-2C.

FIG. 3A is a front view of another embodiment of an ulnar implant of the present disclosure.

FIG. 3B is a front view of yet another embodiment of an ulnar implant of the present disclosure.

FIG. 4A illustrates an elbow in position for implanting an ulnar implant in an elbow joint.

FIG. 4B illustrates exposure of the elbow joint for implanting an ulnar implant in an elbow joint.

FIG. 4C illustrates isolation of the elbow joint for implanting an ulnar implant in an elbow joint.

FIG. 4D illustrates preparation of an ulna with a first tool for implanting an ulnar implant in an elbow joint.

FIG. 4E illustrates further preparation of an ulna with a second tool for implanting an ulnar implant in an elbow joint.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

This application is directed to an elbow joint prostheses and methods that can be used in elbow joint arthroplasty procedures, which can be used to correct elbow joint conditions including, but not limited to, deformity, wear, osteoarthritis, and trauma. As discussed in greater detail below, the apparatuses and methods herein reduce risk of disengagement, dislocation and decoupling, and also facilitate implantation and removal of the apparatuses during surgical procedures. The apparatuses and methods can provide ranges of sizes to better fit a full range of patients. The implants disclosed herein could be made to fit specific patients, e.g., by analysis of imaging of a patient's elbow joint and by configuring the implant to fit with little or no modification of the ends of the bones at the joint.

### I. Elbow Joint Prostheses for Native Bone Interaction

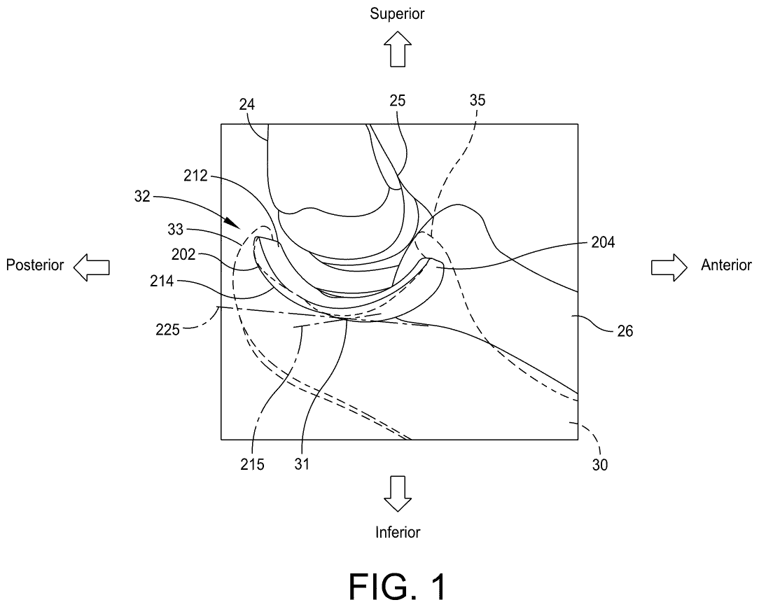

As shown in FIG. 1, the elbow joint can include several bone surfaces, including the end surfaces of the distal humerus 24, the proximal radius 26, and the proximal ulna 30. The distal humerus 24 can include a trochlea 25, which articulates with the ulna 30, and the capitellum, which articulates with the radius 26. The proximal ulna 30 includes a trochlear notch 31 that is configured to receive the medial condyles of the trochlea 25 of the distal humerus 24. The proximal radius 26 includes a radial head that is configured to articulate with the capitellum of the distal humerus 24.

Various embodiments disclosed herein relate to elbow prosthesis assemblies that can beneficially lead to improved patient outcomes, for example, by reducing the volume of bone removed from the patient's humerus, radius, and/or ulna, reducing surgery time, and improving reliability of the prosthesis.

In some conventional full elbow arthroplasty techniques, a humeral stem anchor may be inserted into the patient's distal humerus, a radial stem anchor may be inserted into the patient's proximal radius, and/or an ulnar stem anchor can be inserted into the patient's proximal ulna. The humeral stem anchor can be configured with an articular body to articulate with an articular body of the radial stem anchor and/or an articular body of the ulnar stem anchor. Such stemmed anchors may present long-term fixation issues, as well as undesirable radiologic signatures such as radiolucencies, spot welds, etc. Furthermore, traditional full arthroplasty techniques can fail for a number of reasons, such as dislodgement, early loosening, periprosthetic fractures, infection, instability, and wear. To reduce fixation problems, radiologic signatures, and surgery times associated with traditional stemmed anchors, stemless anchors can be used. Stemless elbow arthroplasty can result in shorter surgery time, less blood loss, and fewer periprosthetic fractures.

Hemiarthroplasty techniques can involve a partial elbow arthroplasty, where only a portion of the elbow joint is replaced. For example, elbow hemiarthroplasty can replace the distal humerus with an implant, but not the radial head or the proximal ulna. The hemiarthroplasty technique can offer several advantages, including retention of native bone, decreased dislodgement, decreased loosening, decreased wear, and decreased fracture of the implant.

Beneficially, various embodiments disclosed herein disclose a stemless implant that preserves native bone for use in subsequent procedures. Moreover, the stemless implants disclosed herein can be biocompatible and can provide beneficial or restorative interactions with native bone. A variety of elbow joint prosthesis assemblies and components are provided herein that include materials to provide enhanced compatibility for native bone interaction. In some cases, the implants described herein comprise materials that foster generation of cartilage on the trochlear or in the joint space between the ulnar implant and the humerus.

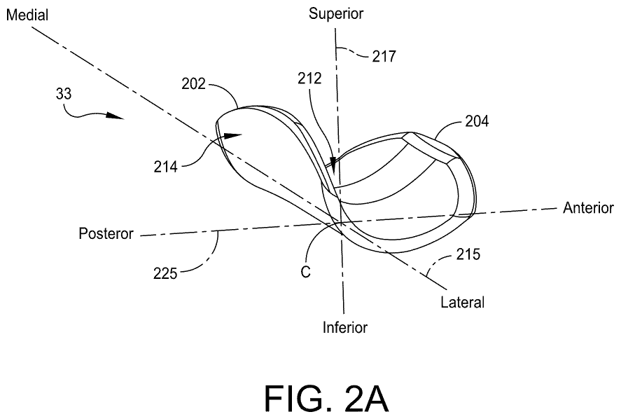

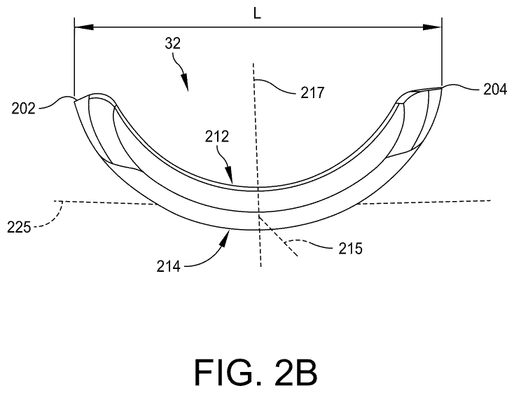

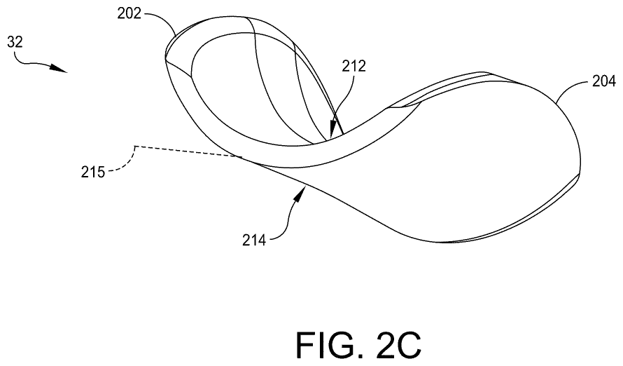

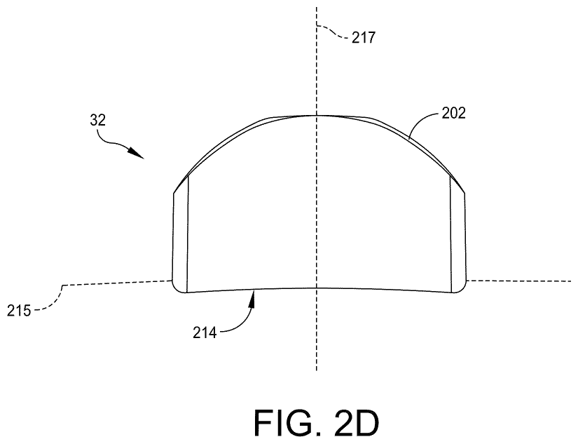

FIG. 1 shows an ulnar implant 32 of the present disclosure for a ulna-humeral hemiarthroplasty, where the ulnar implant 32 is implanted at a proximal end of the ulna 30 and is configured to articulate with the humerus 24. The ulnar implant 32 can be configured to interface with a portion of the native ulna 30 when implanted. In some embodiments, the ulnar implant 32 can be configured to slide over and/or rotate relative to the native humerus 24 after implantation. In some embodiments, the ulnar implant 32 can be configured to slide over and/or rotate relative to a humeral stemmed anchor coupled with the native humerus 24 after implantation. The anatomical directions superior, inferior, anterior, posterior, medial, and lateral are noted in FIGS. 1 and 2A. These directions will be referenced in the detailed description of the configuration of the ulnar implant 32.

In some configurations, the ulnar implant 32 can be a component of an elbow joint assembly configured to interact with other components of the elbow joint prosthesis, such as an articular body of a humeral implant. In other configurations, the ulnar implant 32 can be the entire elbow joint prosthesis and interface directly with the native humerus 24 and the native ulna 30. In some configuration, the ulnar implant 32 is configured to reside between the native ulna 30 and the native humerus 24 without requiring any anchoring components that may extend from or through the thickness of the ulnar implant 32 through a surface layer of the ulna 30, humerus 24 or other anatomy. The ulnar implant 32 can provide smooth sliding or rotating motion with a distal surface of the humerus 24.

The ulnar implant 32 of the present disclosure can be a single piece stemless implant having a curved body. The curved body of the ulnar implant 32 is configured to complement the anatomical shape of the trochlear notch 31 of the ulna 30 on one side, and to complement the anatomical shape of the trochlea of the humerus 24 on the other side. In some examples, the ulnar implant 32 effectively resurfaces the trochlear notch 31 of the ulna 30 and provides the ulna 30 with a new articulating surface that engage the trochlea of the humerus 24. Resurfacing the trochlear notch 31 of the ulna with an ulnar implant 32 can be advantageous in some patients, as compared to replacing the trochlear notch, as it avoids complications such as those related to stability, loosening, or interference with other components.

In some cases, the resurfacing involves leaving some or all of the original surface of the trochlear notch 31 intact. In some cases, the resurfacing involves removing a thickness of the trochlear notch 31 such that the prosthetic trochlear notch surface provided by the ulnar implant 32 can be at the same location as the original native trochlear notch 31. The contralateral trochlear notch 31 can be analyzed to assess whether and how much of the trochlear notch 31 can be removed.

The ulnar implant 32 can be formed following a pre-operative analysis of a particular patient, e.g., following obtaining a CT scan or other imaging of the elbow of a particular patient into which the ulnar implant 32 is to be implanted. The ulnar implant 32 can then be made to an appropriate size for the patient. In some modes, the ulnar implant 32 can be supplied in a kit with a range of configurations for different groups of patients, e.g., different sizes.

As shown in FIGS. 2A-2D, the ulnar implant 32 is a single piece unit forming a curved body extending between a first end and a second end. The first end of the ulnar implant 32 forms a first or posterior edge 202 and the second end of the ulnar implant 32 forms a second or anterior edge 204. The first or posterior edge 202 can be positioned adjacent to or under the olecranon process 33 when implanted in an elbow. The second or anterior edge 204 can be positioned adjacent to or under the coronoid process 35 when implanted in an elbow. The ulnar implant 32 can comprise two major curved surfaces, a first or superior surface 212 and a second or inferior surface 214, extending between the first edge 202 and the second edge 204.

The first surface 212 and the second surface 214 are on opposing sides of the curved body of the ulnar implant 32. The first surface 212 is the surface that complements and engages the anatomical shape of the trochlear notch 31 of the ulna 30 when implanted in a patient. Thus, the first surface 212 is generally concave as shown. The second surface 214 is the surface that complements and engages the anatomical shape of the trochlea of the humerus 24 when implanted in a patient. Thus, the second surface 214 is generally convex as shown.

The result is that the configuration of the ulnar implant 32 is such that the body of the implant 32 is curved away from the posterior edge 202 and the anterior edge 204 of the curved body in the inferior direction. When implanted as intended, the inferior direction would be the direction toward the trochlear notch 31 of the ulna 30.

Referring to FIG. 2A, for purposes of discussion, three orthogonal axes 217, 225, and 215 are defined for the ulnar implant 32. The first axis 217 extends in the superior-inferior direction with respect to the curved body of the ulnar implant 32 and is referred to herein as the S-I axis. Any orientation that is parallel to the S-I axis will be referred to herein as in the S-I direction. The second axis 225 extends in the antero-posterior direction with respect to the curved body of the ulnar implant 32 and is referred to herein as the A-P axis. Any orientation that is parallel to the A-P axis will be referred to herein as in the A-P direction. The third axis 215 extends in the latero-medial direction with respect to the curved body of the ulnar implant 32 and is referred to herein as in the L-M axis. Any orientation that is parallel to the L-M axis will be referred to herein as in the L-M direction. All three axes intersect at a point C that is a geometric center of the ulnar implant 32.

The first surface 212 can be an articulating or articular surface and has a concave curvature in a cross-sectional view when a section is taken along a first plane that contains (i.e., intersects) both the S-I axis 217 and the A-P axis 225. The concave curvature of the first surface 212 in the first plane can be symmetrical or asymmetrical about the S-I axis 217. Additionally, the first surface 212 can have a convex curvature in a cross-sectional view when a section is taken along a second plane that contains (i.e., intersects) both the A-P axis 225 and the L-M axis 215. The first surface 212 is configured in this configuration to articulate or interact with a portion of a native humerus 24 or humeral implant. For example, the first surface 212 can be shaped to maintain contact with a native or prosthetic humeral trochlea of the humerus 24.

The second surface 214 can be a mounting surface, opposite the first surface 212. The second surface 214 has a convex curvature in a cross-sectional view when a section is taken along the first plane that contains both the S-I axis 217 and the A-P axis 225. The convex curvature of the second surface 214 in the first plane can be symmetrical or asymmetrical about the S-I axis 217. Additionally, the second surface 214 can have a concave curvature in a direction orthogonal to the first plane, in other words, in a cross-sectional view when a section is taken along the second plane that contains (i.e., intersects) both the A-P axis 225 and the L-M axis 215. The second surface 214 is configured in this configuration so that the contour of the second surface 214 engages a trochlear notch 31 of an ulna 30 and contributes to maintaining the ulnar implant 32 in place in the elbow joint space between the proximal end of the ulna 30 and the distal end of the humerus 24.

As shown in FIG. 2D, in some embodiments, the second surface 214 has a concave curvature in the L-M direction, viewed in a section taken along the second plane that contains both the A-P axis 225 and the L-M axis 215. In some embodiments where the ulnar implant 32 is configured to be free-floating in the elbow joint space between the distal end of the humerus and the proximal end of the ulna, the concave curvature can have a radius of curvature consistent with the anatomy of the proximal end of the ulna to allow the second surface 214 to follow the curvature of the trochlear notch 31 in the L-M direction.

Referring to FIG. 2B, the ulnar implant 32 has a length L, measured along a straight line from the first edge 202 to the second edge 204, of at least about 30 mm and/or less than or equal to about 40 mm. The length L may be measured along the straight line that intersects the geometric center of the first edge 202 and the geometric center of the second edge 204.

The first edge 202 and/or the second edge 204 of the ulnar implant 32 can be constrained by portions of the trochlear notch 31 in proximity of those edges, which prevents unwanted displacement of the ulnar implant 32 in the A-P direction and/or an S-I direction. Further, the ulnar implant 32 can be positioned at the proximal end portion of the ulna 30, such that the trochlea 25 of the distal humerus 24 at least partially constrains the ulnar implant 32 in place.

In some embodiments, the ulnar implant 32 can be inlayed into the trochlear notch 31 of the ulna 30, such that the ulna 30 can hold the ulnar implant 32 along the periphery surfaces of the ulnar implant 32. When the ulnar implant 32 is implanted, the first edge 202 and the second edge 204 can be held in place by edges of a trochlear notch 31 of the ulna 30. For example, the first edge 202 and the second edge 204 of the ulnar implant 32 can be held in place by a coronoid process 35 and an olecranon process 33 of the ulna 30. Further, because the ulnar implant 32 can be used in a hemiarthroplasty, the soft tissues may be retained, which would further limit displacement the ulnar implant 32. Where multiple sizes of the ulnar implant 32 are provided, the tension in the soft tissue may be one factor in considering the appropriate size of the implant.

In embodiments where the ulnar implant 32 is configured to be inlayed into the proximal end of the ulna, the concave curvature can be defined to provide a surface that would match the prepared surface of the bone at the proximal end of the ulna 30.

In the embodiments where the ulnar implant 32 is inlayed in the ulna, the inlayed configuration provides the ulnar implant 32 a four-way stability, such as stability in both the A-P direction and the L-M direction. The curvatures of the first surface 212 and/or the second surface 214 along the first plane described above can provide stability of the implanted ulnar implant 32 in the A-P direction. The curvatures of the second surface 214 along the second plane described above can provide stability of the implanted ulnar implant 32 in the L-M direction. Therefore, the ulnar implant 32 can be stable at least in a four-way direction (e.g., in both the A-P direction and the L-M direction). The curvature of the second surface 214 along the second plane enables the second surface 214 to provide enhanced position retention function, such that the radius of curvature of the surface 214 in the second plane is smaller than that of the trochlear notch 31 in the L-M direction. The curvature about the second axis 225 also enables a more stable ulnar implant 32.

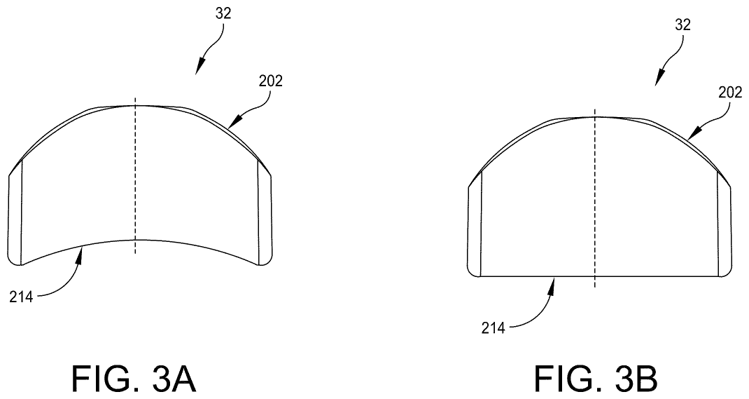

As shown in FIG. 3A, in some embodiments, the concave curvature of the second surface 214 along the second plane may be deeper with a smaller radius of curvature. In some examples, the curvature of the second surface 214 along the second plane has a radius of curvature between about 75 mm and about 150 mm, such as between about 100 mm to about 125mm.

As shown in FIG. 3B, in some embodiments, the second surface 214 can also be flat in the L-M direction. The flat surface may be provided for ease of manufacturing and/or to provide additional strength.

In some embodiments, the curved body of the ulnar implant 32 can be consistent in thickness or width along its length, measured from the first edge 202 to the second edge 204, of the ulnar implant 32. Alternatively, in some embodiments, the curved body can vary in thickness or width along the length, measured from the first edge 202 to the second edge 204, of the ulnar implant 32. For example, the curved body of the implant can be thinner in a central portion compared to the two end regions near the first edge 202 and the second edge 204. In some configurations, a minimum thickness of the curved body can be centered or positioned off-center and closer to one edge of the ulnar implant 32 than the other end of the ulnar implant 32. Alternatively or additionally, the curved body can be thinner towards the first and second ends 202, 204.

The first surface 212 can be configured to articulate or interact with a portion of a humerus 24, which may be a native humerus or humerus implant. The second surface 214 can be shaped to maintain contact with the humerus 24 as the ulnar implant 32 rotates about the first axis 215. For example, the first surface 212 can have a radius of curvature, in the first plane, of at least about 10 mm and/or less than or equal to about 30 mm, such as between 15 mm and about 20 mm, between about 20 mm and about 25 mm, or between about 25 mm and about 30 mm. The radius of curvature of the first surface 212 can be constant or can vary along the length of the first surface 212. When the ulnar implant 32 is designed to interface with a prosthetic implant, the radius of curvature of the first surface 212 in the first plane can have a shallower curve with a radius of curvature between about 20 mm to about 30 mm. When the ulnar implant 32 interfaces with the native bone, the radius of curvature of the first surface 212 in the first plane can have a deeper concave profile with a radius of curvature of at least about 5 mm and/or less than or equal to about 15 mm, for example between 5 mm and about 10 mm.

The first edge 202 and second edge 204 of the ulnar implant 32 can each be thicker than the region near the center region between the first edge 202 and the second edge 204. For example, the ulnar implant 32 can have a maximum thickness, measured at the first edge 202 and/or the second edge 204, between 20 mm and 30 mm. The ulnar implant 32 can have a minimum thickness, measured in the center region, between 10 mm and 20 mm. The minimum thickness region of the implant 32 can be centrally located between the first edge 202 and the second edge 204 or can be off-center.

In some configurations, the first edge 202 and the second edge 204 can be positioned at least 180 degrees or greater from each other, such as between about 180 degrees and about 220degrees. In some configurations, the first edge 202 and the second edge 204 can be less than or equal to about 180 degrees from each other, for example between about 120 degrees and 180degrees.

In some configurations, the ulnar implant 32 can be symmetrical, for example for ease of manufacture. In other configurations, the ulnar implant 32 can be asymmetrical. For example, the first edge 202 and the second edge 204 can have different thicknesses, measured from the first surface 212 to the second surface 214. The first edge 202 can have a first thickness that is larger than the second thickness of the second edge 204. In other configurations, the first edge 202 and the second edge 204 can have similar or equal outer thicknesses. The first edge 202 and second edge 204 can each have a rounded edge, a sharp edge, chamfered edge, beveled edge, or other edge.

In some configurations, the first surface 212 and/or the second surface 214 can each have a smooth surface, a textured surface, or a combination of smooth and textured surfaces. A textured surface can provide friction between the ulnar implant 32 and a native bone to provide additional stability. A smooth surface can promote motion of the ulnar implant 32 and a native bone, which can promote cartilage growth. For example, the first surface 212 can have a textured surface to provide stability between the ulnar implant 32 and the ulna 30, and/or the second surface 214 can have a smooth surface to promote motion between the ulnar implant 32 and the humerus 24.

As illustrated, the ulnar implant 32 may be stemless to promote movement of the ulnar implant 32 against both the ulna 30 and the humerus 24 when the ulnar implant 32 is not inlaid into the proximal end of the ulna 30 to stimulate fibrous cartilage growth. The ulnar implant 32 may be stemless for ease of manufacture. Alternatively, the ulnar implant 32 may also have one or more stems (not shown) to fix the ulnar implant 32 to the native bone and provide stability. The stem can be positioned to be inserted into the ulna in use, e.g. on the side labeled the second surface 214 of the ulnar implant 32.

The ulnar implant 32 may include a material suitable for articulation with native bone, such that the material has near natural wear characteristics, along with an elastic modulus that closely matches cortical bone. For example, the ulnar implant 32 can include or be coated with a material, such as pyrocarbon, synthetic cartilage, or any other material with favorable tribological properties with native bone. For example, pyrocarbon may stimulate tissue (e.g., cartilage) growth upon movement over a native bone surface. Alternatively or additionally, the ulnar implant 32 can include various other materials, such as graphite, carbon fiber, titanium, stainless steel, plastic, other polymeric material, or other suitable biocompatible material. In some configurations, the ulnar implant 32 can have a graphite core coated at least partially or entirely with pyrocarbon on an outer surface or on all outer surfaces. In some configurations, the ulnar implant 32 can be made entirely of or coated entirely with pyrocarbon.

A kit can include a plurality of, e.g., at least two or at least four, ulnar implants 32 of various sizes. The various ulnar implants 32 permit the implantation of an ulnar implant 32 that matches the patient's anatomy. More or fewer than four sizes can be provided. Elbow joint prosthesis kits, according to some examples, may include multiple ulnar implants 32 of different sizes to better fit a full range of patients. In some examples of a kit, the ulnar implant 32 comes in varying sizes, such as with different lengths, heights or widths, or radii of curvature. The various ulnar implants 32 permit the implantation of an ulnar implant 32 that matches the patient's anatomy.

The ulnar implant 32 for the elbow joint prostheses may be selected and implanted according to a range of one or more sizes for a given corresponding humerus or ulna. For example, a kit may include four different sizes of ulnar implants 32, such as small, medium, large, or extra-large ulnar implants 32. The different sizes of ulnar implants 32 permit the ulnar implants 32 to correspond with the respective humerus or ulna in the elbow joint.

### II. Implantation Methods for Elbow Joint Prostheses for Native Bone Interaction

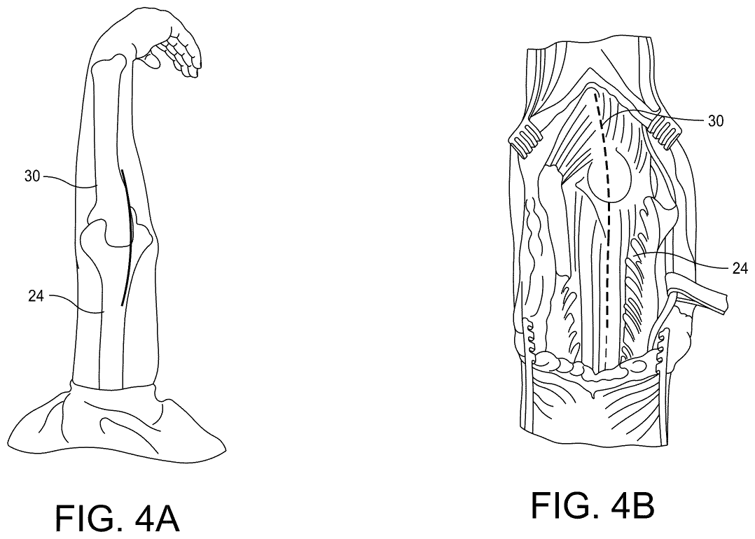

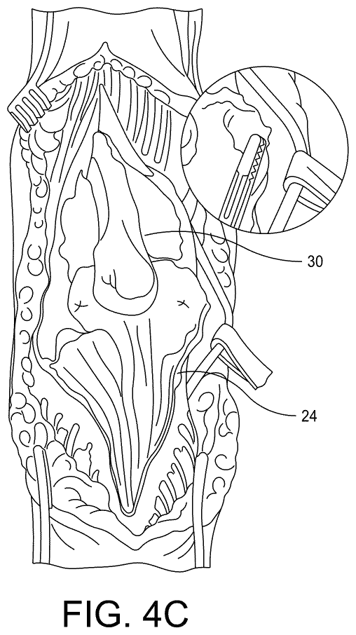

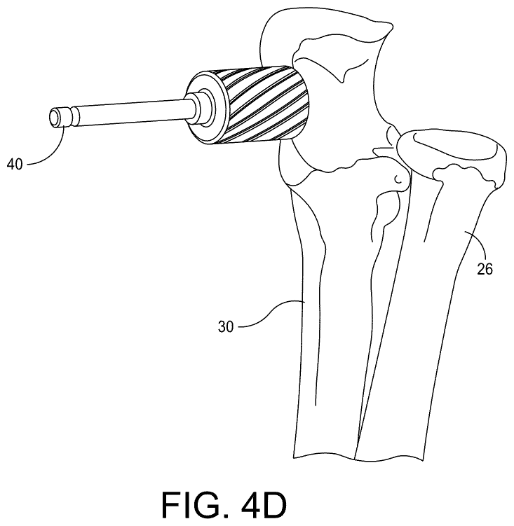

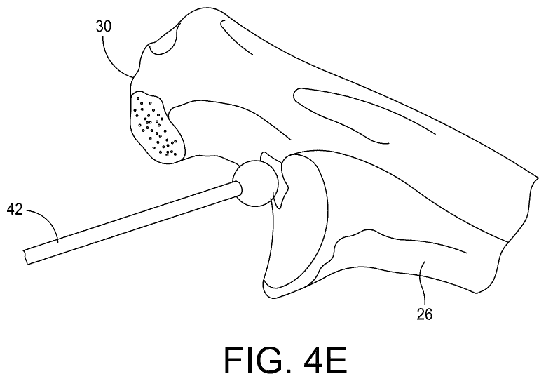

FIGS. 4A-4E show aspects of surgical methods related to the implantation of the ulnar implants 32 as disclosed herein.

In preparation for the surgical procedure, the elbow can be in a lateral decubitus position or a supine position for implanting a humeral implant in an elbow joint (see FIG. 4A). After the elbow is in position, a straight incision can be created along the length of the arm to expose the elbow joint (see FIG. 4B). The tissue, such as the ligaments, muscle, and fascia, can be removed to expose or isolate the humerus 24 and the ulna 30 (see FIG. 4C). The bones of the elbow joint may also be isolated from the nerves so the nerves may be protected during the surgical procedure.

After the elbow joint has been isolated, the end portion of the ulna 30 can be accessed and prepared to receive an ulnar implant 32. As shown in FIG. 4D, a portion of the proximal ulna 30 may be shaped or removed with a first tool 40, such as with a drill or reamer. As shown in FIG. 4E, a portion of the proximal ulna 30 may be further shaped or removed with a second tool 42, such as with a burr or flexible reamer. In some techniques, an instrument can be used to create a negative of the ulnar implant 32 in the proximal ulna 30, such as a recess shaped to receive the ulnar implant 32 in the proximal end portion of the ulna 30 or a C-shape in the proximal ulna 30 to receive the ulnar implant 32. The ulnar implant 32 can then be placed or positioned in the proximal ulna 30 where the bone was removed, such as shown in FIG. 1. In the methods, the elbow joint may be separated or dislocated during the procedure, but the position of the ulnar implant 32 will be in the proximal ulna 30 after the procedure.

Once implanted, the ulnar implant 32 may be constrained by bone and tissue, but allowed to articulate with the humerus 32. The ulnar implant 32 can be positioned at the end of the ulna 30, such that an articular surface of the first surface 212 is configured to rotate relative to the humerus 24. The ulnar implant 32 can be positioned at the end of the ulna 30, such that a trochlea 25 of the humerus 24 at least partially constrains the ulnar implant 32 in place and/or soft tissues of the elbow joint at least partially constrains the ulnar implant 32. In some techniques, the ulnar implant 32 can be positioned such that the ends 202, 204 of the curved member 210 are held in place by edges of a trochlear notch 31 of the ulna 30. For example, the ends 202, 204 of the curved member 210 can be held in place by a coronoid process 35 and an olecranon process 33 of the ulna 30. When implanted, the ulnar implant 32 may also be able to move relative to the ulna 30 as well. The motion of the ulnar implant 32 relative to the ulna 30 can promote or stimulate growth of cartilage.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±1%, ±5%, ±10%, ±15%, etc.). For example, “about 15 mm” includes “15 mm.”

Although certain embodiments and examples have been described herein, it will be understood by those skilled in the art that many aspects of the delivery systems shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

1. An ulnar implant comprising:
a curved body having a first end and a second end, the curved body comprising:
a first surface; and
a second surface, wherein the first surface and the second surface are on opposing sides of the curved body;
wherein the first surface has a concave curvature in a cross-sectional view when a section is taken along a first plane that intersects both S-I axis and A-P axis, wherein the S-I axis is an axis that extends in superior-inferior direction with respect to the curved body of the ulnar implant and the A-P axis is an axis that extends in antero-posterior direction with respect to the curved body of the ulnar implant;
wherein the second surface has a convex curvature in a cross-sectional view when a section is taken along the first plane, and
wherein when the ulnar implant is implanted at an end portion of an ulna, the first surface of the ulnar implant articulates with a portion of a humerus.

2. The ulnar implant of claim 1, wherein the first surface has a convex curvature in a cross-sectional view when a section is taken along a second plane that intersects both the A-P axis and L-M axis, wherein the L-M axis is an axis that extends in latero-medial direction with respect to the curved body of the ulnar implant.

3. The ulnar implant of claim 1, wherein the second surface has a concave curvature in a cross-sectional view when a section is taken along a second plane that intersects both the A-P axis and L-M axis, wherein the L-M axis is an axis that extends in latero-medial direction with respect to the curved body of the ulnar implant.

4. The ulnar implant of claim 1, further comprising a stem for fixation of the ulnar implant at the end portion of the ulna.

5. The ulnar implant of claim 4, wherein the stem is positioned on the second convex surface.

6. The ulnar implant of claim 1, wherein the first surface comprises a radius of curvature between 20 mm and 50 mm.

7. The ulnar implant of claim 1, wherein the second surface comprises a radius of curvature between 20 mm and 50 mm.

8. The ulnar implant of claim 1, wherein the first surface is smooth and the second surface is smooth.

9. The ulnar implant of claim 1, wherein the ulnar implant has a length between 30 mm and 40 mm.

10. The ulnar implant of claim 1, wherein the ulnar implant has a maximum thickness between 20 mm and 30 mm.

11. The ulnar implant of claim 1, wherein the concave curvature of the first surface in the first plane is symmetrical about the S-I axis.

12. The ulnar implant of claim 1, wherein the concave curvature of the first surface in the first plane is asymmetrical about the S-I axis.

13. The ulnar implant of claim 1, wherein the convex curvature of the second surface in the first plane is symmetrical about the S-I axis.

14. The ulnar implant of claim 1 wherein the convex curvature of the second surface in the first plane is asymmetrical about the S-I axis.

15. The ulnar implant of claim 1, wherein the curved body comprises pyrocarbon.

16. A method for treating an elbow joint, comprising:
accessing an end portion of an ulna;
positioning an ulnar implant comprising a curved body at the end portion of the ulna, wherein the curved body having a first end and a second end, the curved body comprising:
a first surface; and
a second surface, wherein the first surface and the second surface are on opposing sides of the curved body;
wherein the first surface has a concave curvature in a cross-sectional view when a section is taken along a first plane that intersects both S-I axis and A-P axis, wherein the S-I axis is an axis that extends in superior-inferior direction with respect to the curved body of the ulnar implant and the A-P axis is an axis that extends in antero-posterior direction with respect to the curved body of the ulnar implant;
wherein the second surface has a convex curvature in a cross-sectional view when a section is taken along the first plane,
wherein when the ulnar implant is implanted at an end portion of an ulna, the first surface of the ulnar implant articulates with a portion of a humerus; and
positioning the first surface of the ulnar implant at the end portion of the ulna to articulate with a portion of a humerus.

17. The method of claim 16, further comprising forming a recess shaped to receive the ulnar implant in the end portion of the ulna.

18. The method of claim 16, further comprising positioning the ulnar implant at the end portion of the ulna, such that a trochlea of the humerus at least partially constrains the ulnar implant in place.

19. The method of claim 16, further comprising positioning the ulnar implant at the end portion of the ulna, such that soft tissues of the elbow joint at least partially constrains the ulnar implant in place.
